(19) United States
(12) Patent Application Publication
(10) Pub. No.: US 2017/0030758 A1
(43) Pub. Date: Feb. 2, 2017

(54) FLUID OUTPUT MEASUREMENT DEVICE AND METHOD

(71) Applicant: Output Medical, Inc., Chicago, IL (US)

(72) Inventor: Jay Joshi, Chicago, IL (US)

(21) Appl. No.: 15/230,713

(22) Filed: Aug. 8, 2016

Related U.S. Application Data
(63) Continuation of application No. 14/738,297, filed on Jun. 12, 2015.
(60) Provisional application No. 62/011,111, filed on Jun. 12, 2014.

(51) Int. Cl. G01F 23/00; A61B 5/20; G06F 19/00; A61B 10/00; G01F 3/00; G05D 7/06

(52) U.S. Cl. CPC G01F 23/0061; G01F 3/00; G05D 7/0635; G06F 19/3406; A61B 10/007; A61B 5/208

(57) ABSTRACT

A fluid measurement device includes a container configured to contain a volume of fluid. The container defines an inlet and an outlet. A plurality of sensors are operatively coupled to the container. The plurality of sensors are configured to detect a fluid level within the container. A processing device is operatively coupled to the plurality of sensors. The processing device is configured to process data transmitted by the plurality of sensors to determine at least one rate-based property relating to the fluid.

### CROSS-REFERENCE TO RELATED APPLICATIONS

This application claims the benefit of the filing date of U.S. Provisional Application No. 62/011,111 entitled “Fluid Output Measurement Device and Method” filed Jun. 12, 2014, the disclosure of which is incorporated by reference herein.

### STATEMENT REGARDING FEDERALLY SPONSORED RESEARCH

N/A

### BACKGROUND

The subject matter disclosed herein relates to a fluid measurement device and associated methods for measuring, recording, analyzing, and evaluating fluid collected by the fluid output measurement device.

Human body fluid output measurements and analysis are essential to clinical management and translational research. Patient care requires diligent evaluation of output and analysis thereof in order to assess optimal fluid balances, loss of fluid, and sources and causes of the fluid output and/or fluid loss.

Most patient fluid outputs are measured by ancillary medical staff in rudimentary containers. The containers have set markings that correspond to a particular output and require analysis done at a separate lab. The medical staff responsible for measuring fluid output compares the fluid output with the markings on the container to ascertain, to the best of their ability, the volume of fluid in the container.

For example, urine output is an important vital sign used in treating patients with Acute Kidney Injury (AKI). In-hospital acquired AKI can be a cause of increased morbidity and mortality among critical care patients. Various clinical studies suggest a direct correlation between the mortality of AKI and the number and duration of low urine output episodes. These studies show that patients who develop in-hospital AKI are at more than three times higher risk of death than patients who do not develop in-hospital AKI.

Currently, urine output in Intensive Care Unit (ICU) patients is measured in hourly intervals (often in intervals of 4 hours) through a transparent, pliable plastic bag or container. Though suitable for some purposes, such an approach does not necessarily meet the needs of all application settings and/or users. For example, this method can be inaccurate, resulting in reduced detection of low urine output episodes. Also, ICU nurses can spend 5%-7% of their time measuring and recording urine output, and these time-intensive tasks can result in higher inaccuracies and/or error. Further, failing to detect the complications of AKI can lead to additional costs per patient, which are absorbed by the hospital in most cases.

### SUMMARY

In one aspect, a fluid measurement device includes a container configured to contain a volume of fluid. The container defines an inlet and an outlet. A plurality of sensors are operatively coupled to the container. Each of the plurality of sensors is configured to detect a fluid level within the container. A processing device is operatively coupled to the plurality of sensors. The processing device is configured to process data transmitted by the plurality of sensors to determine at least one rate-based property relating to the fluid.

In another aspect, fluid measurement device includes a container configured to contain a volume of fluid. The container defines an inlet and an outlet. A proximal valve is positioned at the inlet of the container. The proximal valve is movable between an open position providing fluid communication between a device input tubing and the container and a closed position to prevent fluid from flowing into the container. A distal valve is positioned at the outlet of the container. The distal valve is movable between a closed position to retain the fluid within the container and an open position to allow the fluid to exit the container. A processing device is operatively coupled to the proximal valve and the distal valve to control dispensing of the fluid from within the container.

In yet another aspect, a method includes collecting a volume of fluid in a container, detecting by one or more sensors the volume of fluid in the container, and determining by a processing device coupled to the one or more sensors at least one rate-based property relating to the volume of fluid using sensor data transmitted from the one or more sensors.

### BRIEF DESCRIPTION OF THE DRAWINGS

The detailed description is set forth with reference to the accompanying drawings. The use of the same reference numbers in different figures indicates similar or identical items or features. Various embodiments in accordance with the present disclosure will be described with reference to the drawings, in which:

FIG. 1 is a contextual view of an exemplary fluid measurement device in accordance with various embodiments;

FIG. 2 is a schematic view of a portion of an exemplary fluid measurement device in accordance with various embodiments;

FIG. 3A is a detailed view of an exemplary fluid measurement device in a vertical orientation in accordance with various embodiments;

FIG. 3B is a detailed view of an exemplary fluid measurement device in a non-vertical orientation in accordance with various embodiments;

FIG. 4 is a screenshot of a display for an exemplary software application in accordance with various embodiments;

FIG. 5 is a flow diagram of an exemplary method used with the fluid measurement device in accordance with various embodiments; and

FIG. 6 is a processor platform that may be used to execute machine-readable instructions to implement the embodiments disclosed herein.

The embodiments disclosed herein are not intended to limit or define the full capabilities of the device. It is assumed that the drawings and depictions constitute exemplary embodiments of the many embodiments of the device and methods.

### DETAILED DESCRIPTION

As clinical management evolves, a novel method of output measurements and analyses improves clinical care through superior clinical outcomes and cost savings. This device enables more accurate, cost-effective, and precise output measurements through, at least, the following advantages: removing or limiting human involvement in the measuring process; enabling real-time fluid output measurements; providing more accurate fluid output readings; and creating a modality to integrate fluid output readings with other vital sign readings.

The trends in healthcare and other fields favor a measurement approach that is automated, integrated, and requires less human intervention. This disclosure addresses two key additional trends necessitating even greater need for the disclosed device and corresponding methods: cost efficiency in healthcare, among other fields; and the rise of translational research for biomarkers. Healthcare costs have risen exponentially relative to other fields. It is imperative that devices, such as the device disclosed herein, facilitate the reduction of these rising costs. Additionally, no device exists currently that enables real-time, point-of-care biomarker analysis in the fluid output. Monitoring and measuring changing biomarker levels, particularly when correlated with fluid output as can be achieved by the device described in the present disclosure, has the ability to improve the real-time decision making for physicians in early detection of diseases such as AKI.

This disclosure is not limited to urine output, and the embodiments described herein are suitable for collecting, measuring, recording, analyzing, and evaluating any transudate, exudate, or organic-based body fluid that exits the body, including cerebrospinal fluid, blood loss, chest tube output, peritoneal fluid output, and any histologically-based fluid serving a homeostatic purpose. The disclosure also describes the relationship of a specific fluid output or flow with a specific biomarker, be it a ratio, corresponding trend, or cross-referencing data points. The benefits remain the same—improvements in measurement efficiency and accuracy lead to improved quality of care, reduced hospital costs and improved medical staff productivity, thereby establishing the strong value proposition of the device and corresponding methods disclosed herein.

A fluid measurement device and corresponding methods to measure, record, study, evaluate, and assist in the measurement of, analysis of, and decision-making based upon a specific fluid output are described in various embodiments herein. The fluid measurement device includes a container. One or more sensors are operatively coupled to the container and configured to measure fluid output in real-time, or at discrete intervals, and provide relevant information including, but not limited to, solute concentration, molecular concentration, temperature, volume, and/or flow rate or change in volume. The measurements can take place with the fluid measurement device in multiple orientations, such as a substantially vertical orientation or a non-vertical orientation. In one embodiment, the measurements can be taken with no calibration requirements or additional manipulations that would otherwise be considered excessive.

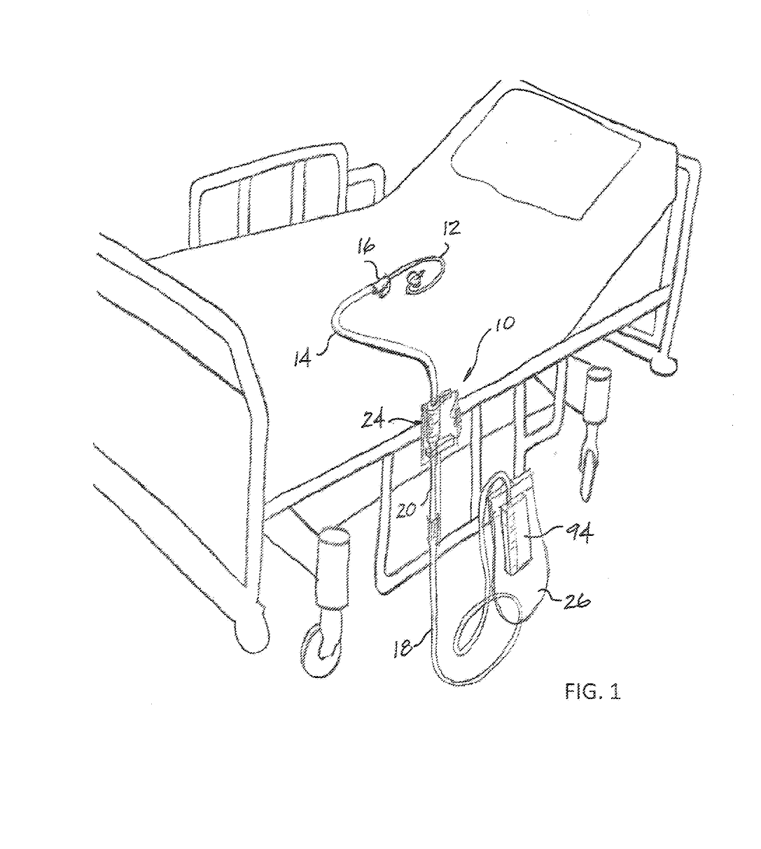

Referring now to the figures, FIG. 1 depicts an exemplary context in which a fluid measurement device 10 is used in accordance with various embodiments. In one embodiment, a catheter 12 including suitable tubing, such as a Foley catheter tubing, is inserted at a first end into a genital orifice for fluid communication with a patient's bladder (though other suitable means of coupling to a body are contemplated as are appropriate in other given settings and applications). An opposite second end of catheter 12 connects to an optionally larger device input tubing 14 that may be an integral part of the fluid measurement device 10 disclosed herein. In other embodiments, the tubing may insert into other parts of the human body, orifices, or surgical sites to collect and measure body fluid output. In a particular embodiment, a sterile seal surrounding a connection 16 coupling the catheter 12 and the device input tubing 14 in fluid communication can be perforated when necessary. In one embodiment, each of the device input tubing 14 and the catheter 12 has a length of 12 inches to 24 inches, for example, in order to accommodate patient movement in the bed without the exercise of tension or sudden pulling on the fluid measurement device 10. In alternative embodiments, however, each of the device input tubing 14 and the catheter 12 may have any suitable length and/or diameter as necessary or desired.

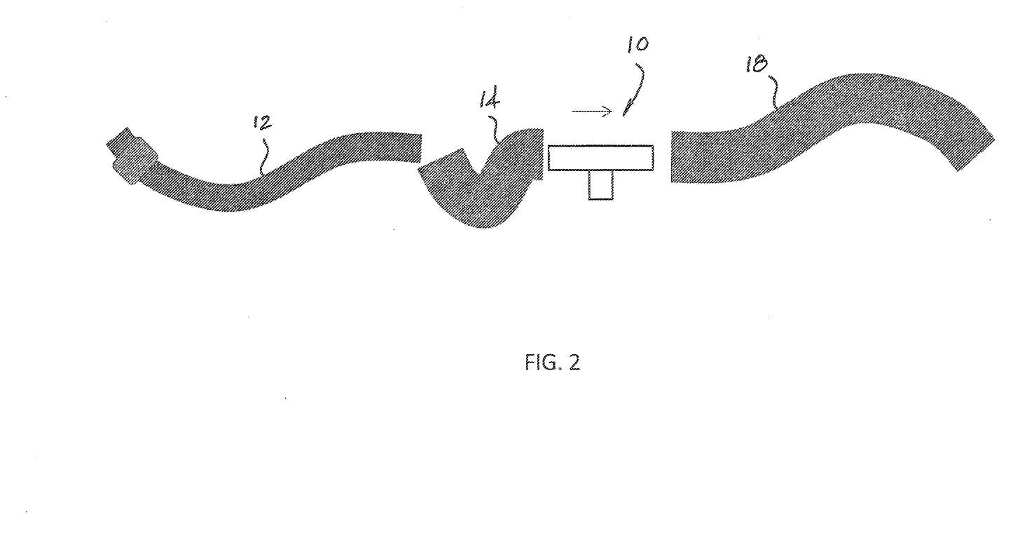

The fluid measurement device 10 is operatively positioned between the catheter 12 and a collection container tubing 18, as shown in FIGS. 1 and 2. The catheter 12 may be a Foley catheter or another suitable catheter, such as, but not limited to, a Jackson-Pratt drain, a pleural tube, or a cerebrospinal fluid tube. Fluid output by the patient generally passes in one direction through the fluid measurement device 10, from the catheter 12 through the device input tubing 14 into the fluid measurement device 10 and discharged or released through a device output tubing 20. In one embodiment, in inserting the fluid measurement device 10, a connection between the catheter 12 and the collection container tubing 18 is separated and the fluid measurement device 10 is manually inserted therebetween. The catheter 12 is then connected to the device input tubing 14 of the fluid measurement device 10 and the device output tubing 20 of the fluid measurement device 10 is connected to the collection container tubing 18.

With continued reference to FIG. 1, in one embodiment, the collection container tubing 18 may be included or may be coupled to the device output tubing 20 by means of one or more suitable couplings and/or one or more suitable seals as are understood in the art. In some conventional contexts, the collection container tubing 18 may have bending and/or kinking, thus proving the need for more proximal measurements in order to mitigate measurement errors, such as fluid retention within the collection container tubing 18. Additionally, a proximal location of the fluid measurement device 10 to the fluid source improves flow rate calculations and increases the measurement accuracy.

In one embodiment, one or more portions or the entire fluid measurement device 10 are disposable. For example, portions of the fluid measurement device 10 that may or may not contact the fluid may be disposable while certain other portions not contacting the fluid may be non-disposable. In another embodiment, the entire fluid measurement device 10 is disposable. In yet another embodiment, portions of or the entirety of the fluid measurement device 10 may be included as part of a fluid output draining mechanism comprising of a Foley catheter set (or tray) or another drainage set, including, but not limited to, a Jackson-Pratt drain, a pleural tube, or a cerebrospinal fluid tube. By this, sterility can be maintained without having to manually insert the fluid measurement device 10.

Returning now to FIG. 1, the fluid measurement device 10 may include a hook 24 or a place-guard allowing the fluid measurement device 10 to be anchored to a supporting structure, such as a hospital bed, in a substantially upright, vertical orientation with respect to a support surface, such as the building floor. The hook 24 can be easily and securely affixed to any structure, platform, or cross-rail found commonly in the in-patient setting. One purpose of the hook 24 may be to prevent or minimize movement of the fluid measurement device 10 to non-vertical orientations. In various embodiments, the fluid measurement device 10 can be included with the device input tubing 14 and/or the device output tubing 20 separate from the fluid measurement device 10, positioned at or near a collection container 26, or attached as a separate add-on container.

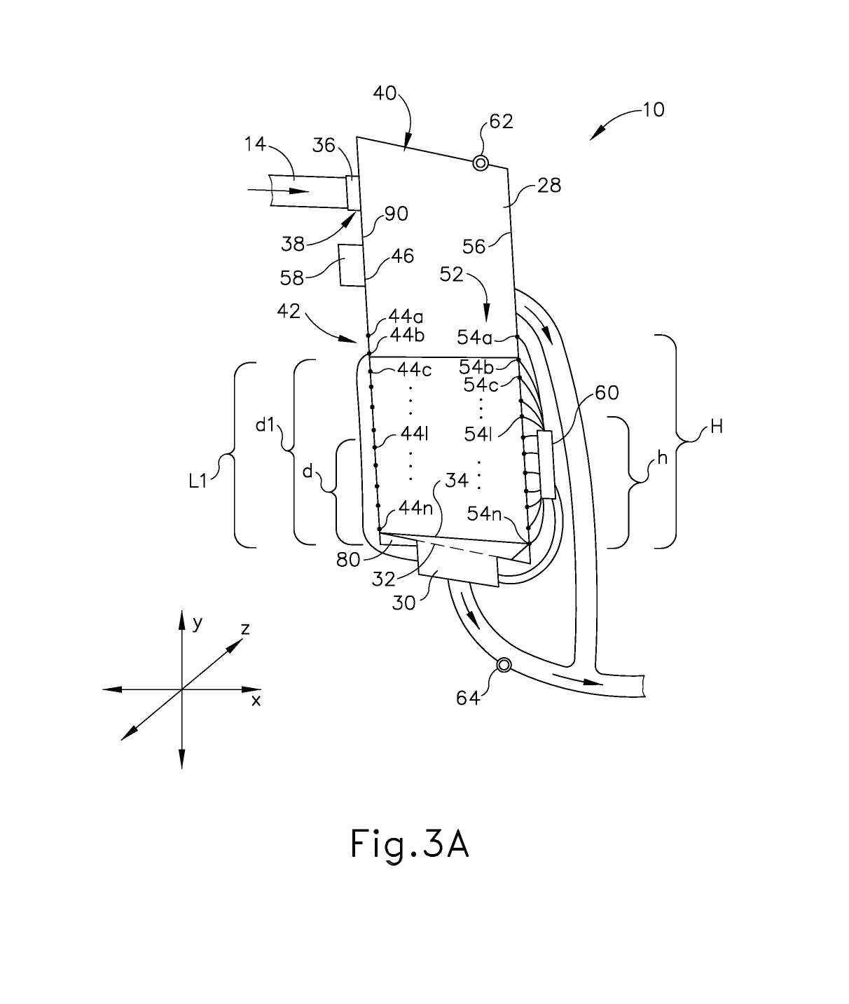

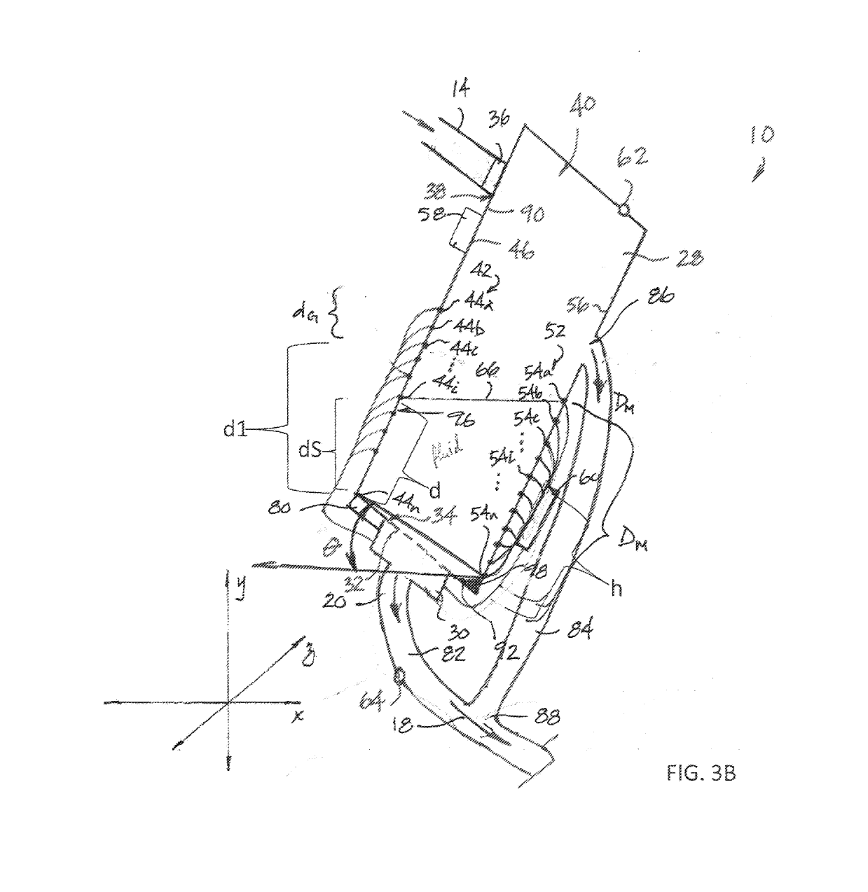

Turning now to FIGS. 3A and 3B, a more detailed description of the exemplary fluid measurement device 10 is provided in accordance with various embodiments. Given the typical usage of fluid output collection and measurement devices in hospital environments it is desirable that the fluid measurement device 10 can operate accurately in a wide range of orientations as described by its rotation within a reference frame commonly described by the three axes of rotation x, y, and z. FIG. 3A shows an exemplary fluid measurement device 10 in a vertical orientation while an exemplary non-vertical orientation is depicted in FIG. 3B. For clarity, it is beneficial to first describe how the fluid measurement device 10 operates in a strictly vertical orientation where its axes of symmetry are aligned with those of the reference coordinate frame shown in FIGS. 3A and 3B. Referring to FIG. 3A, the fluid measurement device 10 includes at least one container 28 with a first or distal valve 30, such as a suitable release valve, positioned at an outlet 32 defined by the container 28 at or near a first or bottom edge or surface 34 of the container 28. Fluid enters the fluid measurement device 10 through the device input tubing 14 which, in one embodiment, is an integral part of the fluid measurement device 10 but need not be. In certain embodiments, a second or proximal valve 36 is positioned at an inlet 38 defined by the container 28 at or near a second or top edge or surface 40 of the container 28 to control, in cooperation with the distal valve 30, fluid input into the container 28 and/or fluid output from the container 28, as described in greater detail below. The terms “top” and “bottom” as used herein to identify the edges or surfaces of the fluid measurement device do not necessarily refer to a direction referenced to gravity. In certain embodiments, the container 28 defines a suitable volume in the range of 5 milliliters (ml) to 100 ml, for example, to enable the fluid measurement device 10 to hold or contain and measure fluid volumes in the range of 1 ml to 50 ml, for example. The size of the container 28, in one embodiment, is 1 inch (in) to 5 inches in the maximum dimension with a larger dimension along the vertical axis than along the other two axes in order to minimize measurement errors. In alternative embodiments, the container 28 may have any suitable size, shape, and/or configuration to define any suitable volume for containing a desired volume of fluid.

When the distal valve 30 is in a closed position, fluid accumulates in the container 28 and a level of fluid within the container 28 may increase. A plurality of sensors are operatively coupled to the container 28, and each sensor is configured to detect a fluid level within the container 28. A processing device is operatively coupled to the plurality of sensors, and configured to process data transmitted by the plurality of sensors to determine at least one rate-based property relating to the fluid. In one embodiment, an exemplary first series or array 42 of any suitable number of sensors 44a, 44b, 44c, . . . 44i, . . . 44n capable of detecting a presence of fluid are aligned along an inside surface or an outside surface of the container 28, or within a wall 46 of the container 28. In the embodiment shown in FIGS. 3A and 3B, a corresponding second series or array 52 of sensors 54a, 54b, 54c, . . . 54i, . . . 54n capable of detecting the presence of fluid are aligned opposite corresponding sensors 44a, 44b, 44c . . . 44i, . . . 44n along an inside surface, an outside surface or within a wall 56 of the container 28. Alternative embodiments may include one or more suitable sensors.

In one embodiment, one or more of the sensors 44a, 44b, 44c, . . . 44i, . . . 44n and/or one or more of the sensors 54a, 54b, 54c, . . . 54i, . . . 54n are capacitive sensors. The first array 42 of sensors and/or the second array 52 of sensors may be incorporated in a thin, flexible circuit to accommodate a curved surface of the container 28. Alternatively, one or more of the sensors 44a, 44b, 44c, . . . 44i, . . . 44n and/or one or more of the sensors 54a, 54b, 54c, . . . 54i, . . . 54n may be embedded on a suitable printed circuit board (PCB) for mounting on a flat surface. In a particular embodiment, the sensors 44a, 44b, 44c, . . . 44i, . . . 44n and/or the sensors 54a, 54b, 54c, . . . 54i, . . . 54n include embedded software which can be configured either for auto-calibration for ease of use or manual calibration to maximize the accuracy.

While one exemplary embodiment for the placement of the sensors 44a, 44b, 44c, . . . 44i, . . . 44n and/or the sensors 54a, 54b, 54c, . . . 54i, . . . 54n is described with reference to FIGS. 3A and 3B, the sensors 44a, 44b, 44c, . . . 44i, . . . 44n and/or the sensors 54a, 54b, 54c, . . . 54i, . . . 54n can be arranged in multiple patterns—random, defined by trigonometric or other non-linear mathematical functions, or any combination thereof. The sensors 44a, 44b, 44c, . . . 44i, . . . 44n and/or the sensors 54a, 54b, 54c, . . . 54i, . . . 54n may be arranged in a manner to optimize the accuracy of the measurements and/or to optimize the cost of manufacturing, including using the fewest sensors possible. The size, orientation, and/or proximity of adjacent sensors are intended to minimize error from measurements. For instance, two possible sources of error from the placement of the sensors are: a distance between the adjacent sensors and the size of the sensors. For example, the sensors 44a, 44b, 44c, . . . 44i, . . . 44n may be positioned in very close proximity to minimize errors due to fluid in the space between adjacent sensors remaining unaccounted. Additionally, the focus of the sensor placement and patterns may not be to increase the accuracy of all measurements, but to increase the accuracy of a set of measurements, within a defined range, and/or at fixed volumes. By setting baseline values of optimal measurement ranges, optimal measurement intervals are achieved as opposed to optimal measurements overall. One or more additional sensors can be placed with respect to the device input tubing 14 proximal to the container 28 and/or with respect to the device output tubing 20 distal to the container 28 and/or with respect to a bypass channel (described below) of the container 28.

Various embodiments of the fluid measurement device 10 may incorporate multiple sensor types. These sensors can detect, measure, and/or analyze relevant information from the fluid, including, without limitation, information related to total volume, rate, solute concentration, analyte, compound, temperature, density, and/or opacity of the fluid and/or of the substances and solutes within the fluid. Information obtained from the sensors may correlate to other clinical data as well. For example, sensors placed within the container 28 or on an outer surface of the container 28 may detect clinically relevant information about the fluid output, including the volume, rate, concentration, analyte presence, temperature, density, and/or opacity of the fluid and/or of the substances and solutes within the fluid. Sensors may include, without limitation, one or more of the following: resistive, capacitive, ultrasound, and/or thermal sensors, or any combination thereof.

In addition to sensors for measuring and analyzing fluid output, the fluid measurement device 10 may include one or more sensors 58 that independently monitor an orientation of the fluid measurement device 10 and that can detect rapid motions such as jerking motions or other random movements. For example, one or more accelerometers may be operatively coupled to the container 28 to detect such aberrant motions and transmit this information to a controller, as described below, so that appropriate error control algorithms can be applied in order to reduce or eliminate the influence of sudden motions on the sensor states and, therefore, volume calculations. Further, in one embodiment the sensors 44a, 44b, 44c, . . . 44i, . . . 44n and/or the sensors 54a, 54b, 54c, . . . 54i, . . . 54n are configured to differentiate readings that are due to aberrant motion of fluid within the container 28 relative to actual filling differences by introducing a suitable time lapse between detecting the fluid and transmitting a signal to the controller indicating that the sensor is in an “on” state.

Once one or more sensors, for instance sensor 44i and/or the sensor 54i shown in FIG. 3A, at a certain height h from the bottom edge 34 of the container 28 senses the presence of fluid at that height, a state of the sensor 44i and/or the sensor 54i changes from “off” to “on,” and a volume of the fluid in the container 28 based on the height h and the geometrical dimensions of the container 28 can be readily computed and recorded by a processing device, such as a suitable controller 60. A time elapsed since the last time the container 28 was empty can also be recorded. In a particular embodiment, the controller 60 may adjust the volume reading to account for meniscus formation based on the container geometry and readily available formulae. When the fluid level reaches the sensor 44a and/or the sensor 54a positioned farthest from the bottom edge 34 of the container 28 at a height H, the sensor 44a and/or the sensor 54a transmits a corresponding signal to the controller 60 that a maximum allowable capacity of the container 28 has been reached and the controller 60 in turn activates the distal valve 30 to open to release the fluid from the container 28. In one embodiment, one or more first air vents 62 are positioned at or near a top portion of the container 28 above the sensors 44a and 54a to allow air flow through (i.e., into and/or out of) the container 28 to facilitate release of the fluid from within container 28. In certain embodiments, one or more additional second air vents 64 are positioned distal to the outlet 32 to facilitate fluid movement through the fluid measurement device 10.

In certain embodiments, only one sensor at a height H is sufficient to measure a certain pre-determined volume, however situating multiple sensors in a vertical line is advantageous as it allows a multiplicity of volumes to be measured and recorded independently of the release of the fluid from the container 28. In other embodiments, the sensor(s), such as the first array of sensors 42 and/or the second array of sensors 52, may be situated in the geometrical middle of the container 28 and attached to a support rod or tube (not shown), extending downwards from the top edge 40 of the container 28 opposite the bottom edge 34. For example, in one embodiment, the fluid measurement device 10 may include a spout (not shown) that assists the fluid that enters into the container 28 from the device input tubing 14 to collect at a bottom portion of the container 28, and the first array of sensors 42 and/or the second array of sensors 52 are coupled to the spout. Any other suitable support structure or structures for attachment of the sensors known in the art may be used within the container 28 in any orientation. FIG. 3A demonstrates one particular embodiment of heights H and h, respectively.

Referring additionally to FIG. 3B, we now consider operation of the fluid measurement device 10 when the fluid measurement device 10 is tilted at an exemplary non-vertical orientation. In certain embodiments, the first array of sensors 42 and/or the second array of sensors 52 are situated at the exact geometrical middle of the container 28 and the container 28 is rigid with a symmetrical shape. In this instance, at any orientation of the container 28 along the axes x, y, and z, the level of the fluid in the container 28 at those sensor(s) locations will not change substantially if the rotation is slow and/or when the system equilibrates at the new orientation. Therefore, with such a placement of the sensor(s) the fluid measurement device 10 is able to measure and record the volume of fluid in the container 28 at a wide range of orientations without sacrificing accuracy.

As shown in FIG. 3B, each of the first array of sensors 42 and/or the second array of sensors 52 are placed on or along opposing walls 46, 56, respectively, of the container 28. In this embodiment, additional sensors and/or computations may be necessary to determine the volume of the fluid within the container 28. The first array of sensors 42 and/or the second array of sensors 52 are placed in a symmetrical configuration along opposing walls of the container 28 such that pairs (or quadruples) of sensors at the same distance (or height if the container 28 is in a vertical orientation) from the bottom edge 34 of the container 28. While only two arrays of sensors are depicted in FIGS. 3A and 3B capturing changes in orientation around the y-axis, it is understood that such or additional arrays of sensors can be placed on other opposing walls of the container 28 in order to capture changes in orientation around the x-axis. If the fluid measurement device 10 is in a vertical orientation as shown in FIG. 3A with the fluid at a certain level L₁, the sensors at a corresponding distance d₁, (where h/d and H/D coincide in this position) from the bottom edge 34 of the container 28, i.e., 44b and 54b, each transmits to the controller 60 a signal indicating a detection of the presence of the fluid; thus, providing an error check for the fluid measurement device 10. Further, in certain embodiments each of the sensors situated at distances d from the bottom edge 34 of the container 28 less than d₁, as shown in FIG. 3A, will also transmit to the controller 60 a signal indicating a detection of the presence of the fluid, thereby building in additional error checking capabilities.

Referring to FIG. 3B, when the fluid measurement device 10 is tilted at a non-vertical orientation different from the vertical orientation shown in FIG. 3A, at least one of the corresponding sensors in the first array of sensors 42 and the second array of sensors 52, i.e., the sensor 44i and the sensor 54i (or quadruples if the tilt is along more than one axis) at the same distance d from the bottom edge 34 of the container 28 may no longer detect the presence of fluid in the container 28. In FIG. 3B, as an example, the two sensors at the same distance d, or paired sensors, include the sensor 44i and the sensor 54i. Instead, as depicted by the line in FIG. 3B representing the fluid level 66, and as an illustrative example, if the sensor 54a on a first side of the container 28 at the greatest distance D_{M}=D from the bottom edge 34 of the container 28 is detecting a presence of the fluid, one or more of the sensors 44a, 44b, 44c, . . . 44i, . . . 44n on the opposing side of the container 28 may remain above the fluid level. Therefore, if the sensor 44c on the second side of the container 28 at a distance d₁ from the bottom edge 34 of the container 28 detects the presence of the fluid, all sensors, i.e., sensors 44a, 44b in the first array of sensors 42 at greater distances d_{G} may not detect a presence of the fluid, while all sensors, i.e., sensors 44d, . . . 44i, . . . 44n in the first array of sensors 42 at shorter distances d_{S} detect a presence of the fluid, as shown in FIG. 3B. The controller 60 processes and records continuously or periodically the state of each of the sensors, and, in the exemplary embodiment described above, determines that the maximum allowable fluid level has been reached with respect to the first side of the container 28 and transmits an operational control signal to the distal valve 30 to open allowing the release the fluid from within the container 28, while at the same time computing a volume V_{T} less than a volume V_{M} when the container 28 is in the vertical orientation. The volume V_{T} is readily computed from the geometry of the container 28 and from an angle of tilt or tilt angle θ. In this example, the tilt angle θ is determined by: 1) an offset in the number of sensors between a top sensor, i.e., sensor 54a on the first side of the container 28 and the corresponding highest activated sensor on the second side, i.e., sensor 44i; and 2) a spacing between the adjacent sensors in the respective arrays.

While FIG. 3B exemplifies only one angle (tilt angle θ) along one axis (x-axis) at which the container 28 may be tilted, it is understood that similar principles of the volume computations apply when the container 28 is oriented at different angles and along two axes and the volumes can be computed by readily available geometrical formulae embedded as software in the controller 60 in certain embodiments. In certain embodiments, rotation around the z-axis does not impact the level of the fluid in the container 28 and therefore will not influence the volume calculations.

In certain embodiments, the controller 60 is in operational control communication with each of the distal valve 30 and the proximal valve 36. For example, the controller 60 may activate the distal valve 30 to open and/or close by transmitting a signal to the distal valve 60 at either fixed intervals or at variable intervals determined by a pre-defined calculation and/or a defined function. In a particular embodiment, as the controller 60 activates the distal valve 30 to open or close, the controller 60 also activates the proximal valve 36 to close or open to facilitate release or discharge of the fluid from within the container 28. As the distal valve 30 opens, the measured and analyzed fluid is released. The controller 60 is configured to control when the distal valve 30 will open, close, and for how long, as well as calibrate the measurements and analysis. Once all the fluid is released from the container 28, the controller 60 is configured to change the status of the sensors 44a, 44b, 44c, . . . 44i, . . . 44n and/or the status of the sensors 54a, 54b, 54c, . . . 54i, . . . 54n from the “on” state to the “off” state. The distal valve 30 and/or the proximal valve 36 can be opened, held open, and closed through mechanical stimulus, electrical stimulus, magnetic stimulus, and/or any other suitable known method or combination thereof. In one embodiment, the distal valve 30 may be a solenoid valve. The duration of the valve opening, the rate of opening and closing, and/or other mechanical factors related to the measurement and analysis accuracy can be adjusted in real-time depending on the volume of fluid to be released from the container 28.

As shown in FIG. 3B, in certain embodiments there may exist an additional flow blocking mechanism, such as the proximal valve 36, at the inlet 38 to the container 28 that prevents or limits fluid from entering into the container 28 while the distal valve 30 is opened to release the measured fluid. In this embodiment, the proximal valve 36 facilitates preventing or limiting unmeasured fluid from passing through the fluid measurement device 10 while the measured fluid is being released from within the container 28. In certain embodiments, the proximal valve 36 is similar to the distal valve 30 at the outlet 32 of the container 28 or can be any suitable valve or mechanism functioning to prevent or limit fluid movement into the container 28 while the distal valve 30 remains open. In one embodiment, the proximal valve 36 may prevent or limit back flow of fluid toward the device input tubing 14 when the fluid measurement device 10 is tilted at extreme angles with respect to the vertical orientation regardless of whether the distal valve 30 is open.

In one embodiment, a valve release mechanism 80 is coupled to an external surface of the container 28 that allows manual release of fluid from the container 28 by setting and adjusting the distal valve 30 in an open position. The valve release mechanism 80 may employ a button, switch, lever, pull-out piston or any other suitable mechanical mechanism known in the art. Additionally, the valve release mechanism 80 may coordinate measurement of fluid output at discrete time intervals that are clinically necessary to optimize real-time decision making for patient care. The manual valve release mechanism 80 may operate both the distal valve 30 and the proximal valve 36 mechanically without requiring external power. Upon activating the valve release mechanism 80, and prior to release of the fluid, an automatic measurement may be generated by coordinating the opening of the distal valve 30 with reading of the status of the sensors prior to the valve opening via the controller 60.

In another embodiment, fluid release and measurement cycles are automated and coordinated by software embedded in the controller 60. For example, in one implementation, measurements and release of fluid may occur simultaneously at discrete time intervals set at times clinically relevant for real-time clinical decision making. These time intervals may be defaulted to reflect national standards of optimal measurement intervals or may be set per the specific clinical caretaker's preferences given appropriately documented clinical need for such a change.

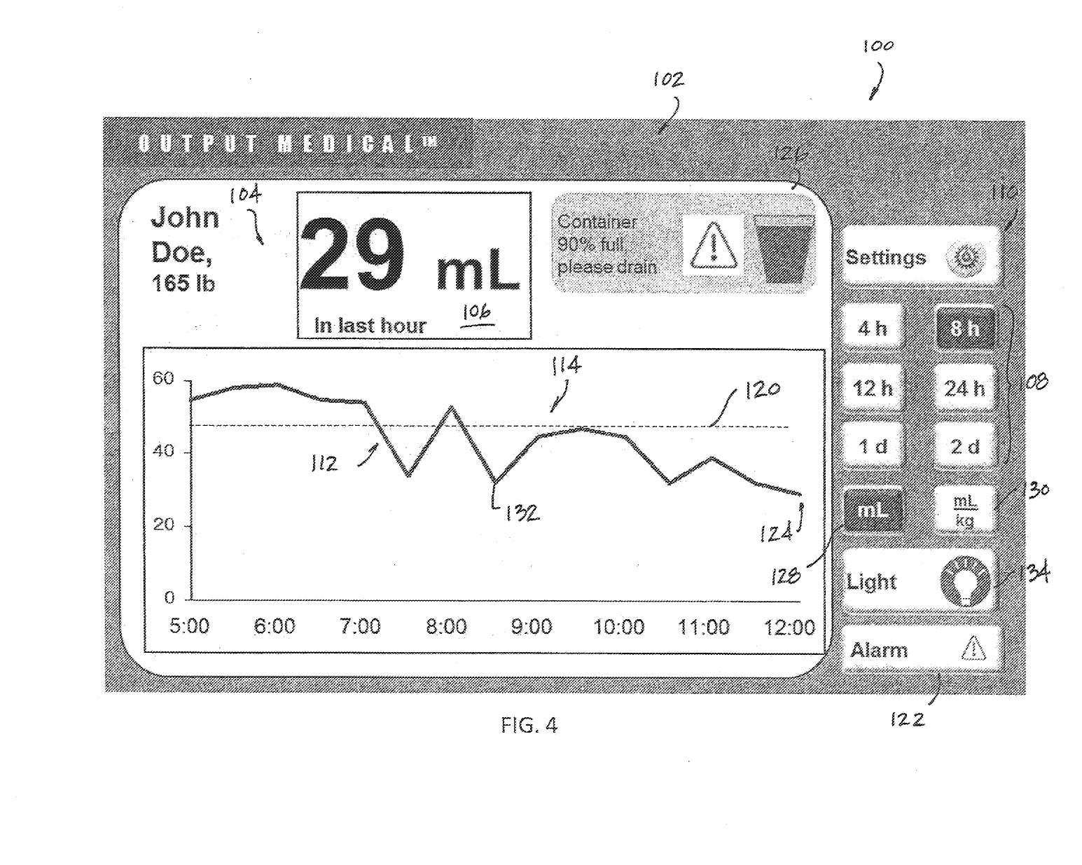

Patients in the clinical setting may present a wide range of fluid outputs around what is considered the normal output as normalized for body weight, or another clinical parameter. For example, whereas some patients may have oliguria associated with very low rates of urine output, other patients may have polyuria which is associated with excessively high levels of urine output. The difference between the low urine output and the high urine output may be as much as one hundred fold. Therefore, it is desirable that the fluid measurement device 10 can operate not only at a wide range of orientations but also at a wide range of flow rates. Thus, in certain embodiments, the fluid level measurement and the fluid release intervals while still simultaneous may be increased or reduced automatically depending on the increased or decreased rates of fluid output observed from the filling rate of the container 28 or by the prior time intervals of fluid release. The controller 60 also communicates the time intervals and the volumes (and/or other properties of the fluid) measured at those time intervals to software for processing and display such as depicted in FIG. 4.

In one embodiment, the fluid measurement device 10 is designed so that the measurement of the fluid volume and other fluid properties does not need to be simultaneous with the release of the fluid from the fluid measurement device 10. By using a multiplicity of sensors communicating with the controller 60 in the manner described above, very frequent measurements of the fluid volume (or other fluid properties) can be recorded along with the times when a given volume (or other fluid property) was measured, thereby enabling computations of the fluid flow rates (or the rates associated with other fluid properties). The release intervals of the fluid from the container 28, however, need not be simultaneous with those of the measurement intervals and may be considerably longer. Uncoupling the fluid measurement and release intervals allows for dynamical adjustments of the collected volume of fluid in the fluid measurement device 10 depending on the rate of fluid inflow and, thus, enables a single container with a fixed volume to measure fluid output at both low flow rates and high flow rates. Therefore, unlike other prior art measurement techniques the fluid measurement device 10 does not need two or more separate containers or a multiplicity of fluid containers within containers to enable the measurement process. In addition, uncoupling the fluid measurement from its release allows for more efficient management of the power requirements, if necessary, to operate the valve.

The fluid measurement device 10 does not require any active pumping or movement of the fluid and only requires the passive inflow of fluid to complete measurements. Additionally, the fluid measurement device 10 may not require a counterweight, or information on additional fluid movement, gravitational restraints beyond ensuring passive fluid movement, heat exchange, or thermal dissipation.

As described above, in one embodiment, the fluid measurement device 10 includes one or more air vents, such as a first air vent 62 at or near a top portion of the container 28 and/or a second air vent 64 positioned at or near a bottom portion of the container 28 as shown in FIG. 3B. In a particular embodiment, the first air vent 62 and/or the second air vent 64 is integral to the fluid measurement device 10. Each of the first air vent 62 and the second air vent 64 is configured to facilitate regulating a pressure in the system by eliminating or reducing positive pressure (“back pressure”) events as well as negative pressure (“suction”) events within the system, and particularly within the container 28, further improving device capabilities and allowing for faster release of the measured fluid from the fluid measurement device 10. The first air vent 62 and/or the second air vent 64 allow air to escape the fluid measurement device 10 to prevent back pressure events and air to enter into the fluid measurement device 10 to prevent suction events. The first air vent 62 and/or the second air vent 64 may be any suitable vents known in the art. In one embodiment, each of the first air vent 62 and/or the second air vent 64 includes a plastic inner membrane (not shown) that will not wet-out during use. The plastic inner membrane also acts as a bacterial and viral barrier with greater than 99.99% efficiency.

In one embodiment, the second air vent 64 prevents or limits air locks that may render the fluid measurement device 10 inoperable or may slow down or decrease a rate of fluid release from the fluid measurement device 10 through the distal valve 30. The airlocks may be created by static pockets of fluid in the collection container tubing 18 which may form from time to time when the tubing forms bends or kinks as a result of the positioning of tubing and/or the collection container 26. The second air vent 64 may enhance the rate of exit of the fluid from the container 28 into a distally-located output channel 82 in fluid communication with the container 28 through the distal valve 30 and in fluid communication with the device output tubing 20 and the collection container tubing 18 in cases where an airlock has formed.

In one embodiment, the fluid measurement device 10 includes a bypass channel 84 incorporated to prevent backflow into the catheter 12 whether due to a sudden excess output of the fluid from the patient that exceeds the available free volume of the container 28 or due to malfunction of the fluid measurement device 10. In case of device malfunction, the bypass channel 84 allows fluid to escape to the collection container tubing 18 and the collection container 26 shown in FIG. 1 in order to prevent backflow of fluid through the catheter 12 and potentially the patient's bladder or fluid accumulation that may cause infections. Fluid can also enter the bypass channel 84 through a secondary outlet 86 if the container 28 is tilted to an extreme non-vertical orientation. The secondary outlet 86 is in fluid communication with the output channel 82 distal to the container 28 at a suitable junction or connector 88.

The outlet 32 of the container 28 through the distal valve 30, the output channel 82 and into the distal device output tubing 20 can be shaped in a manner to prevent or limit stasis of fluid and designed to minimize measurement error in the fluid measurement device 10. In one embodiment, a width of the output channel 82 is set at a specific diameter to minimize an opening time of the distal valve 30 and ensure complete, rapid evacuation of the fluid. The ratio of a diameter of the output channel 82 relative to the distal valve 30 can be a function of the opening time required of the distal valve 30. Restraints on the output channel diameter may be partially or wholly based on the container geometry. The bypass channel 84 and the output channel 82 connect within the fluid measurement device 10 in order to maintain a direction of the fluid flow towards the collection container 26.

In certain embodiments, the combination of the first air vent 62 and/or the second air vent 64 and the bypass channel 84 minimizes or eliminates fluid retention in the fluid measurement device 10, and, particularly within the container 28, and/or backflow into the catheter 12 that may prompt undesirable infections. In one embodiment, the container 28 is designed with a shape that facilitates complete draining of the fluid, for example, narrowing or tapering at or near a bottom of the container 28. Alternatively or additionally, in certain embodiments, one or more components of the fluid measurement device 10, such as an inner surface of the container 28, for example, includes a suitable bactericidal coating 90 or other suitable coating as is known in the art to limit or prevent the risk of contamination and/or infection.

In one embodiment, the fluid measurement device 10 measures one or more biomarkers including, but not limited to, biomarkers that may be indicative of clinical inflammatory responses, lack of responses, clinically significant reactions, and/or clinically important information. For example, for urine output, a clinical response for AKI may be detected by suitable biosensors 92 indicating biomarkers including, without limitation, uNGAL, pNGAL, KIM-1, pCyc, and IL-18. The biosensors 92 that analyze components within the fluid can have associated immunoassays, analyzing a presence and/or a concentration of a particular substance, compound, molecule, and/or complex analyte within the fluid. In one embodiment, the fluid measurement device 10 includes an immunoassay unit or module 94, shown in FIG. 1, in which measurement and analysis can take place and be recorded. These analytes hold relevant information that impact real-time decision making and/or overall informational analysis specific to the fluid. In certain embodiments, the biosensors 92 detect particular molecules, particulates, and/or any clinically relevant organic-based substance within the fluid that identifies important information about the kidney function, for example, and about the overall body function, including, without limitation, cardiac, pulmonary, oncologic, lymphatic, hematological, neurologic, gastrointestinal, hepatobiliary, musculoskeletal, general inflammatory, immunologic conditions, or any combination of these and/or other conditions. In one embodiment, one or more biosensors 92 are located on the inner surface, within, and/or outside of the container 28. The biosensors 92 can multiplex and coordinate information regarding analyte concentration, presence, and/or any changes thereof, and can communicate with a sentinel sensor or microcontroller or display information directly. Fluid output values can be correlated with values and trends in critical biomarkers to enable analysis of fluid output with biomarker values to identify critical trends, ratios, and/or rates to impact clinical decision making.

In one embodiment, corrosion of the sensors 44a, 44b, 44c, . . . 44i, . . . 44n and/or the sensors 54a, 54b, 54c, . . . 54i, . . . 54n and the distal valve 30 can be limited or prevented by an anti-corrosive coating 96 along at least a portion of the inner surface of the container 28. This anti-corrosive coating 96 does not impact overall measurements or analysis. Additionally, one or more suitable sensors can be placed on the external surface of the container 28 or embedded in the walls of the container 28 preventing the need for a corrosive-resistant coating.

Additionally, material within the fluid that may precipitate can be collected and siphoned distally toward the collection container 26. The fluid measurement device 10 can be designed specifically to prevent sediments 98 from the fluid to collect and aggregate at a distal portion of the fluid measurement device 10, and, particularly, at or near the lower portion of the container 28, for example, through the container design and the distal valve orientation and design. Additionally, a coating can be included around the output aspect of the container 28 and the distal valve 30 to further prevent accumulations that can impact device function or measurement accuracy. The shape, contours, and/or design specifications of the container 28 can be adjusted for optimizing discharge or release from the container 28 of different fluids with varying viscosities, output rates, and/or other important fluid characteristics.

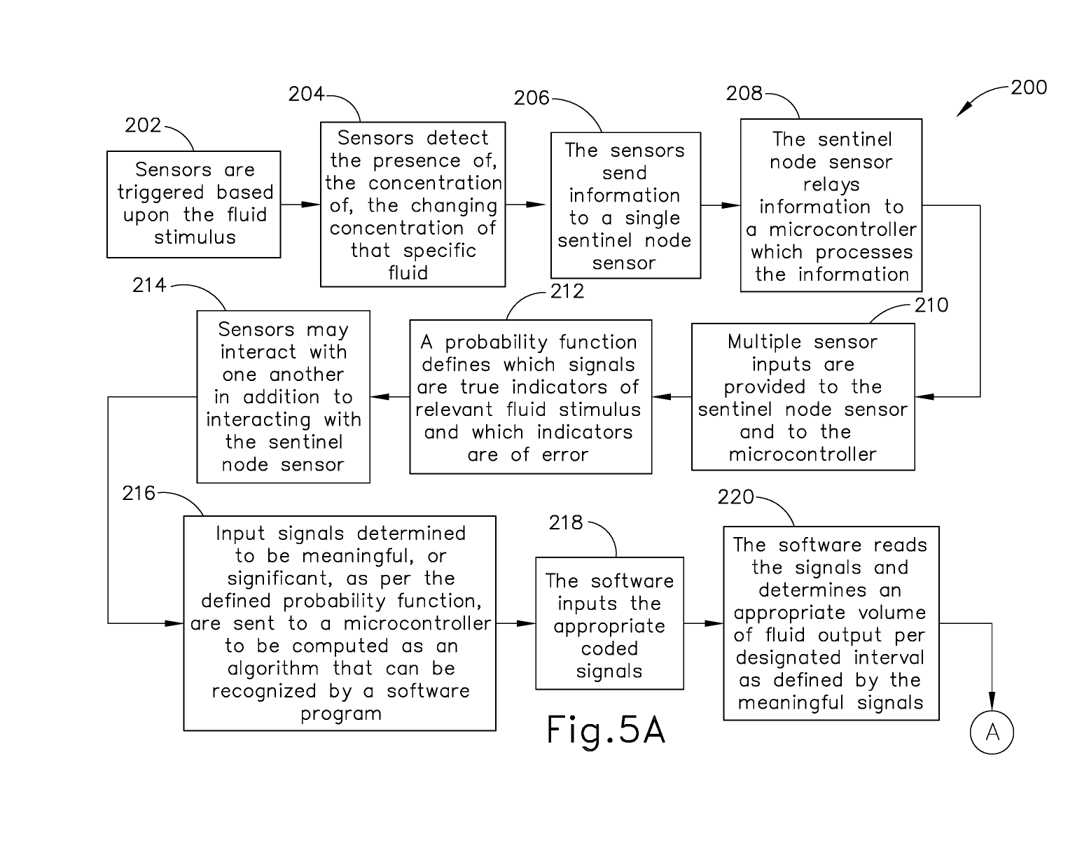

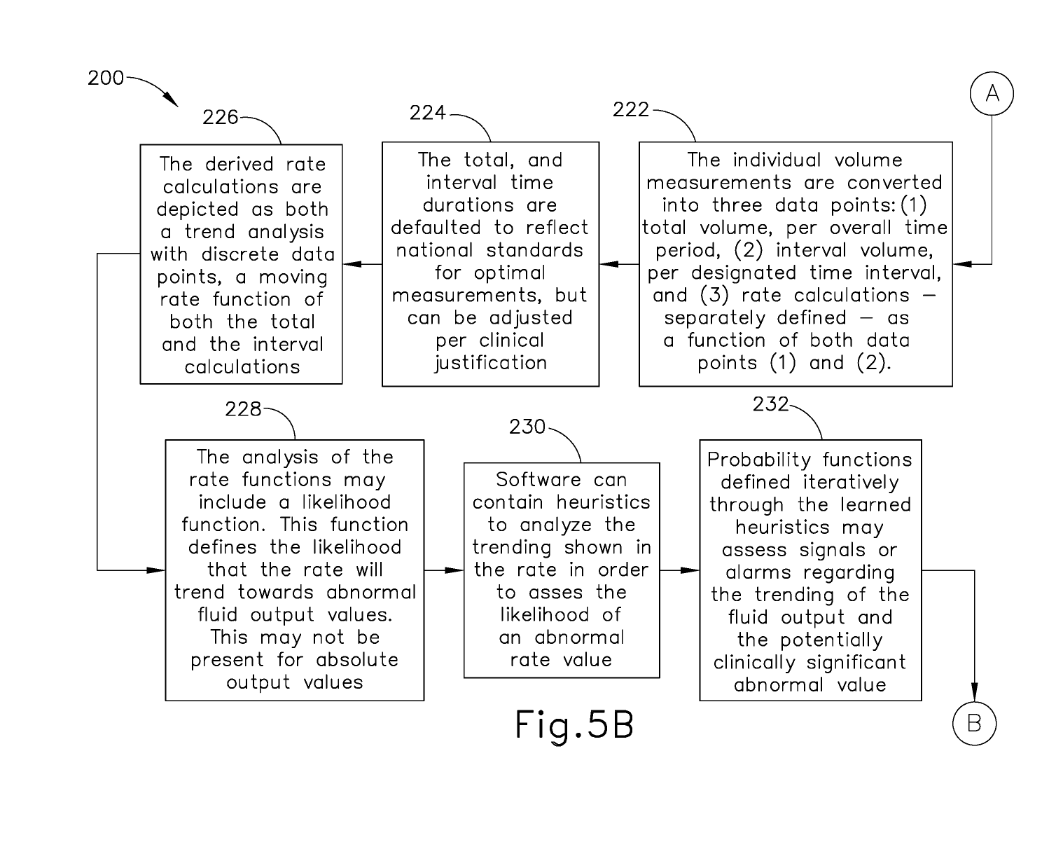

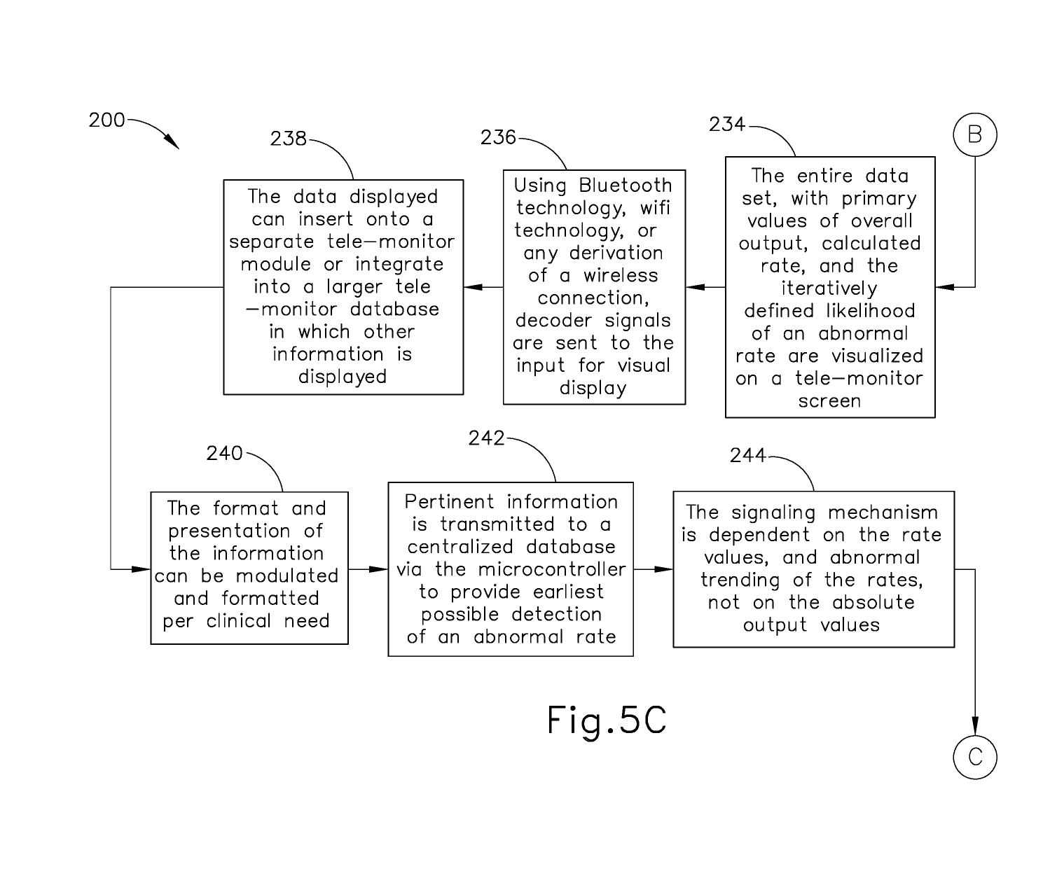

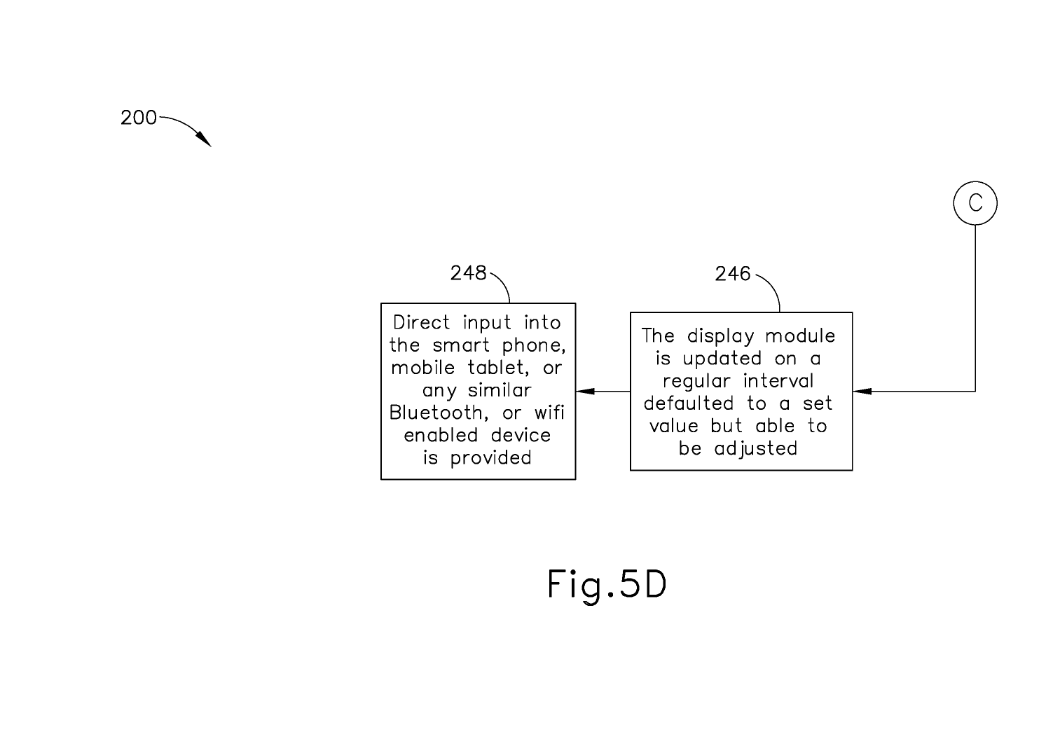

In yet another embodiment, the collection container 26 is positioned at a distal end of the fluid measurement device 10 to collect fluid output from the container 28. The collection container 26 may be any suitable fluid collection container as is known in the art. In a particular embodiment, the collection container 26 itself serves as the fluid measurement device 10, as described in further detail below with reference to FIG. 5.

The fluid measurement device 10 may communicate with one or more software programs that may be configured to display device metrics and information including, for example, properties of the collected fluid. These display units may be independent consoles, integrate into telemetric display units, integrate into an existing computer network, or be displayed upon the fluid measurement device 10 itself. Referring to FIG. 4, an exemplary screenshot 100 of such a display 102 is illustrated. The software system depicted in FIG. 4 includes clinical decision support mechanisms. For example, per the clinical guidelines set by the health caretaker, the software can be configured to provide meaningful data to impact real-time decision making at the point of care. Software can be a specific form of clinical decision support.

The screenshot 100 shown on FIG. 4 can be exhibited on a separate display or integrated within a larger display screen enabling data presentation alongside other key vitals. For example, a dedicated display 102 can be included on or operatively coupled to the fluid measurement device 10. However, the fluid measurement device 10 may be connected to a larger system (such as a computer network, patient care network, electronic medical or health record, a telemetric network, or any patient confidential server intended for clinical support) and will enable display of the pertinent data within a separate window of, for example, a computer display that may be used at a nurses' station or other point of care display device.

The information 104 reported may include the overall fluid output 106 per hour and/or the fluid output per user-defined time interval settings 108 within a range that may be longer or shorter than one hour, with exemplary intervals 110 that can be adjusted from time to time based upon the clinical need defined by the clinical caretaker.

The information reported may include the overall fluid output 106, or the rate of change 112 in fluid output, or other fluid properties calculated using the data points 114 generated by the fluid measurement device 10. The data points 114 may be or may not be independent of the container volume release and the measurement interval per release, and may be calculated and visualized at discrete time intervals 116 chosen at the discretion of clinical caretaker. The data points 114 shown in FIG. 4 may include a finite series of data points that may either be stored or replaced as new data points are generated. The information from the data points may encompass all data points as accrued, or may limit information to only more recent data points displayed as a rolling window graph, and/or a rolling or moving average.

Time intervals may be defaulted to reflect national standards of optimal measurement intervals or may be set per the specific clinical caretaker's preferences given appropriately documented clinical need for such a change, for example, as shown in FIG. 4. The software may include an upper limit and a lower limit for rate calculations and absolute output calculations over a defined time interval that can alert the caretaker if the values fall outside of that range. Software may utilize heuristics to analyze the trend of the rate in order to assess the likelihood that a drop in fluid output or flow below a commonly accepted threshold 120 (or change in another fluid property measured by the fluid measurement device 10) signifies a clinically relevant process such as, but not limited to, AKI. For example, if a patient has a history of reversible drops in fluid output, then upon the recording of a new rate or an absolute output value below the specified lower limit the probability model may predict a low likelihood of AKI or other abnormalities. However, if the patient has a documented history of abnormally low or high rate or absolute output values, captured by the data points, then a new measured abnormal value will be assigned a higher probability when generating a warning signal.

In certain embodiments, the software may also apply the same or different heuristics for the absolute output, correlations with biomarkers, second order and higher rate functions, and trends that analyze the relationship between fluid output and biomarker values and trends, even though these analyses and trends may not be relevant to the clinical diagnosis of abnormal conditions, such as AKI, and may result in information noise and non-relevant clinical data. Learning and heuristics may be incorporated on the likelihood of abnormal values based upon a first-order analysis of the rate, which is analogous to acceleration. Inflection points, change in trends, rate of trends, and/or additional information derived from rate calculations, such as an acceleration of flow or a change in acceleration, may be ignored or assessed less importance, or a weight, in the learning and heuristics. As an example, a visual display of this process is shown in FIG. 4. Alerts 122 can be provided, per a caretaker's discretion, to signal the presence of one or more abnormal values 124, for example an indication 126 that the container 28 is not draining appropriately.

Data can be integrated into a centralized database where the data can be analyzed in real-time along with other vital signs and/or other critical clinical data. Data can be displayed as a fluid output 128, or a fluid output divided by the patient's body mass 130. In certain embodiments, the fluid output information measured by the fluid measurement device 10 is converted into or correlated to point of care information for influencing real-time decision making. Decisions impacted include whether to provide the patient with additional fluid, less fluid, enhance output, restrict output, implement fluid replacement interventions, and/or manipulate fluid spacing in the human body, or other clinically relevant decisions that incorporate the data obtained by the fluid measurement device 10 and a patient's clinical needs. For example, at a discrete point 132, a decision may be made whether to proceed with a specific intervention based on a trending, and a subsequent reverse trending of the fluid output as shown in FIG. 4.

Various display time intervals such as total interval length and/or relative interval length can be managed by input located on the module itself or peripherally, for example, from a centralized database, centralized control, or other remote control, which may include a similar visual format as depicted in FIG. 4.

In one embodiment, the screen position, orientation of data points, overall appearance, and presentation format of FIG. 4 can be adjusted per the caretaker's preference. To adjust the format, one can use vibro-acoustic or touch-screen capabilities to physically slide data displays from one part of the screen to another part. The formatting mechanism can automatically adjust per the change. A light feature 134, such as a backlight, may assist in visualizing the readings without requiring ambient light.

Turning now to FIG. 5, a flow diagram illustrating various features and aspects of a software associated with control of the fluid measurement device 10 and/or reporting of data is illustrated in accordance with various embodiments. The software may be executed in whole or in part across multiple different processors or platforms. For example, a controller including one or more processors may execute portions of software relating to control of the fluid measurement device 10, while other portions relating to display and output of data results may be executed on a different platform, such as a computer. Further, although depicted in one flow chart in FIG. 5, the current disclosure contemplates that various steps can be omitted, added, duplicated, rearranged, or combined with other steps while still within the ambit of the present disclosure.

In the exemplary embodiment shown in FIG. 5, an exemplary method 200 includes at step 202 activating or triggering one or more sensors are based upon a fluid stimulus. If a specific fluid stimulus is recognized by one or more sensors, then a signal will be generated by those sensors indicating activation. Each sensor may detect one or more fluid stimuli and emit different signals in response to each of the stimuli. At step 204, the sensors may then detect a presence of, a concentration of, and/or a changing concentration of a solute or substance such as, but not limited to, an enzyme or a biomarker in the specific fluid. In one embodiment, the sensors cannot emit multiple activation signals at the same time. In this embodiment, if the presence of a particular stimulus is detected, then only that corresponding signal will be transmitted. If the presence of a different stimulus is detected, then that corresponding signal may be transmitted at a discrete time interval subsequent to the transmission of the initial signal.

At step 206, the sensors send or transmit information to a single sentinel node sensor or directly to a microcontroller. The signals are totaled and assessed for each specific stimulus. Information can come from each individual sensor or in aggregate. If the latter, the sentinel node sensor relays the aggregated information at step 208 to a microcontroller which processes the information. If a specific signal indicates a property of the fluid—be it the presence of the fluid or a specific concentration of a substance in the fluid, for example—then the microcontroller can assess the strength of that signal based upon the number of sensors transmitting that signal. Multiple sensor inputs may be provided to the sentinel node sensor and to the microcontroller at step 210. If multiple signals corresponding to a specific stimulus are accumulated, then the total signal is amplified to indicate greater presence of that stimulus. The strength and frequency of the signal or signals can be used to gather additional information about the fluid output and clinical relevance of the signal or signals.

At step 212, a separate probability function can be generated that defines which signals are true indicators of relevant fluid stimulus and which signals are indicative of error. This probability function assigns a likelihood to all inputs derived. The microcontroller then determines which inputs are amplified and which inputs are not through redundancies or aggregated data, but not necessarily limited to these two methods. Based upon the number of redundant signals, the timing, duration, and/or frequency of the signals, among other details in certain embodiments, the signals are determined to be statistically significant as representative of a stimulus and therefore meaningful information. For example, a specific distribution, which can be a Gaussian distribution, a Poisson distribution, or another probability function distribution, can be defined as the appropriate probability function required to determine the significance of each stimulus signal. The specific signal frequency, which can be strengthened through amplification and redundancies, is assigned a probability value to determine meaningfulness. The amplification requires a certain threshold of signal strength in order to distinguish meaningful inputs from noise. Meaningful inputs can take into account all information, and can assign equal or greater importance, or weight, to signals conveying overall information about first-order rates relative to second or higher order rates all of which correspond to fluid flow and broader trend analyses. The concept of first-order and higher order rates relates to the learning and heuristics model, with the critical difference being that the learning and heuristics model seeks to identify future trending and the probability likelihood function seeks to identify the significance of the existing data points. The probability distribution of the likelihood function will be different for each patient. In evaluating this distribution function, the x-axis reflects discrete output values. These values are defaulted to reflect established guidelines for standard values of a specific fluid output, a biomarker, or specific ratio of biomarker to fluid output, but can be adjusted per documented clinical necessity. For example, in measuring urine output, the values would reflect standards of the Acute Kidney Injury Network (AKIN) or a similar organization for oliguria, acute kidney injury and polyuria. The curvature of the distribution would adjust per patient given the past medical history and the ongoing input of new data and new information.

At step 214, sensors may interact with one another. If one sensor is activated with a specific signal function, then other sensors may be prompted to determine the presence of a stimulus, and its significance at step 216. At step 218, input signals determined to be meaningful or significant per the defined probability function are sent to the microcontroller to be computed as an algorithm that can be implemented as a software program. The decoding of the input signals, per the probability function, can take place as the signals are being generated. This probability function takes into account the redundancy, frequency, amplification, and duration of each specific signal. It is not necessary to include both a sentinel sensor and microcontroller.

The software reads and inputs the appropriate coded signals at step 218. The software may include HL-7 compatibility to allow integration of all data sources and to allow output formatting into multiple software platforms in turn. If an appropriate signal is transmitted, then the software interface will identify how to convert the signals into output data. The software reads the signals and determines an appropriate volume of fluid output or other fluid property per designated interval as defined by the meaningful signals at step 220. If the software is able to determine the appropriate output based on the signals, then the data can be visually displayed.

At step 222, the individual volume (or other properties of the fluid as may be substituted below instead of volume) measurements may then be converted into three data points: (1) total volume per overall time period; (2) interval volume per a designated shorter time interval; and (3) rate of volume change separately defined as a function of both data points (1) and (2). The total volume, the interval volume, and/or the rate of volume change may be displayed in separate areas of the monitor. The total volume represents the fluid output since the measurements began. The interval volume is determined by the exact time interval that is measured. The moving rate functions, calculated from data points (1) and (2), can accrue all data points or compute moving or rolling averages as new data points stemming from the meaningful signals are obtained.

At step 224, the total time duration and the interval time duration may be defaulted to reflect national standards for optimal measurements, but can be adjusted per clinical justification. If the intervals are adjusted, then the values will reiterate and adjust based upon the ongoing signal inputs. The derived rate calculations can be depicted at step 226 as both a trend analysis with discrete data points and a moving rate function of both the total and the interval calculations, as depicted in FIG. 4. The most recent data points and the overall trending data points can be displayed.

As shown in step 228, the analysis of the rate values may include a learning and heuristics function. This function defines the likelihood that the rate will trend towards abnormal fluid output values, a trend that may not be evident when analyzing absolute output values. The function is primarily utilized as an instrument to assess repeatability of abnormal values.

At step 230, the software may include a heuristics function to analyze the trending in the rate in order to assess the likelihood that a rate value is abnormal. If the patient had a prior history of abnormal values, then the software will assess for a repeat pattern and acknowledge a higher likelihood that a given measured value is abnormal. In one embodiment, the software does not predict or diagnose abnormal values, but assesses the repeatability of abnormal values based upon the iteratively defined heuristics algorithm. The likelihood that an event can repeat enables the clinician or the caretaker to determine what appropriate clinical interventions, or lack of interventions, are needed. At step 232, probability functions defined iteratively through the learned heuristics function assesses signals and/or alarms regarding the trending of the fluid output and the potentially clinically significant abnormal values. If the signals detected an abnormal rate, then the trending of the rates—based upon a function that incorporates signal frequencies, amplifications, and redundancies—will be seen as a potential repeat event. Potential repeat events are then monitored and reported. The reporting mechanism integrates into a centralized database allowing the caretaker and the clinician to document the event.

In step 234, in certain embodiments, the entire data set, or at least a majority of the data set, with primary values of overall output, the calculated rate, and the iteratively defined likelihood of an abnormal rate, is visualized on a display screen, as illustrated in FIG. 4. If an abnormal trend is likely to appear, then the visual display can indicate a warning and transmit a warning signal telemetrically to the centralized database and telemetric unit. Using Bluetooth technology, Wi-Fi technology, or any suitable derivation of a wireless connection, for example, at step 236, the microcontroller transmits decoder signals to a visual display. If a centralized database exists for telemetric monitoring, then the system can integrate into that database enabling real-time point of care information. At step 238, the data displayed can insert onto a separate display module or integrate into a larger display database in which other information is displayed. At step 240, the format and presentation of the information can be modified and formatted per clinical need.

In step 242, pertinent information is transmitted to a centralized database via the microcontroller or via other routes to provide an earliest possible detection of an abnormal rate. If the microcontroller determines that a measured rate is abnormal, then the microcontroller integrates the prior information with the present information to provide a comprehensive array of information enabling the caretaker to make the most appropriate clinical decision. As a result, the clinician or the caretaker can determine the appropriate clinical intervention having full access to all clinical information, optimizing clinical decision making. The order of information or the format of the information presented to the clinician or the caretaker can be set to a default standard that can be adjusted to the clinician's or the caretaker's preference in order to maximize the efficacy of the information generated. As indicated in step 244, in one embodiment, the alarm mechanism is dependent on the rate values and abnormal trending of the rates rather than on the absolute output values. Noise in the data is eliminated when generating and transmitting a signal, such as a warning signal. In certain embodiments, the current disclosure focuses on the rate of change in fluid output as the clinically relevant information.

At step 246, the fluid property information shown on the display module is updated at regular time intervals that default to a set value but can be adjusted. In step 248, direct input into a smart phone, mobile tablet, or any similar Bluetooth or Wi-Fi enabled device, for example, is provided. The clinician or the caretaker can determine what information he or she wishes to receive and how that information will be presented.

Many of the functional units described in this disclosure have been labeled as modules, devices, software, or other discrete nomenclature in order to more particularly emphasize their implementation independence. For example, a module may be implemented as a hardware circuit comprising custom VLSI circuits or gate arrays, off-the-shelf semiconductors such as logic chips, transistors, or other discrete components. A module or software may be implemented in programmable hardware devices such as field programmable gate arrays, programmable array logic, programmable logic devices or the like.

Modules may also be implemented in software for execution by various types of processors. An identified module of executable code or other portions of software may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, software or a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. The data collected may reference a specific fluid flow or output, a specific biomarker, or a ratio or relationship between a fluid output and biomarker.

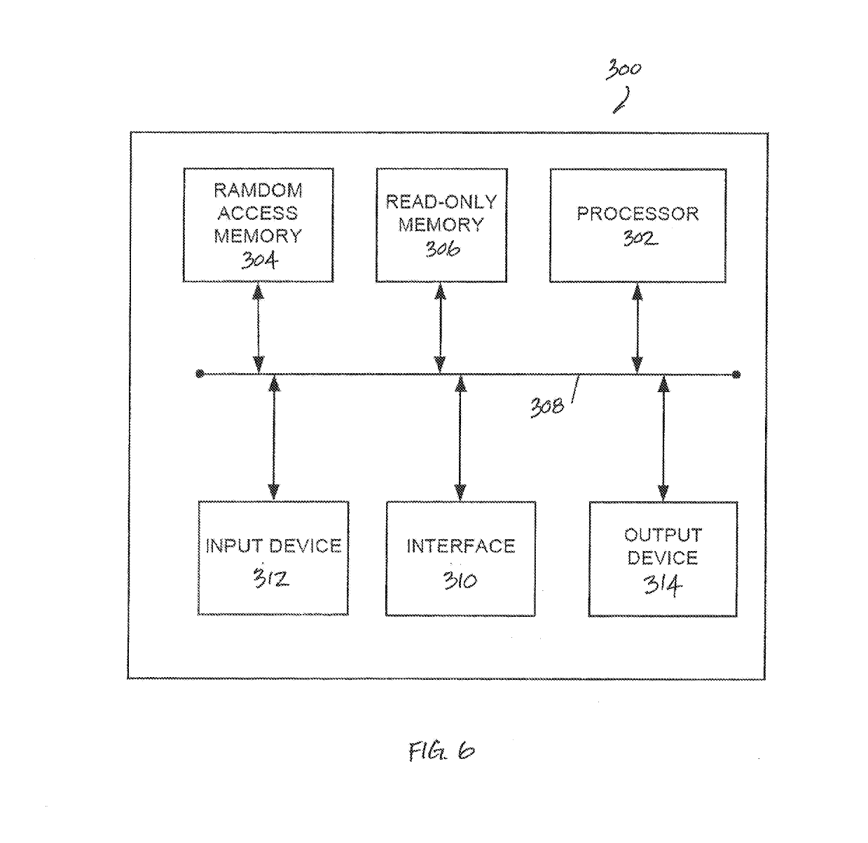

FIG. 6 is a schematic view of an exemplary processor platform 300 that may be used to execute instructions to implement the method 200 of FIG. 5 to implement the fluid measurement device 10 shown in FIGS. 1, 2, 3A and 3B, and the software application shown in FIGS. 4 and 5. In some embodiments, the processor platform 300 is implemented via one or more general-purpose processors, processor cores, microcontrollers, and/or one or more additional and/or alternative processing devices.

The processor platform 300 of FIG. 6 includes a programmable, general purpose processor 302. The processor 302 executes coded instructions within a random access memory 304 and/or a read-only memory 306. The coded instructions may include instructions executable to implement the method 200 of FIG. 5. The processor 302 may be any type of processing device, such as a processor core, a processor and/or a microcontroller. The processor 302 is in communication with the random access memory 304 and the read-only memory 306 via a communications bus 308. The random access memory 304 may be implemented by any type of random access memory device such as, for example, DRAM, SDRAM, etc. The read-only memory 306 may be implemented by any type of memory device such as, for example, flash memory. In some embodiments, the processor platform 300 includes a memory controller to control access to the random access memory 304 and/or the read-only memory 306. The processor platform 300 of FIG. 6 includes an interface 310. The interface 310 may be implemented by an interface standard such as, for example, an external memory interface, a serial port, a general-purpose input/output, and/or any other type of interface standard. The processor platform 300 of FIG. 6 includes at least one input device 312 (e.g., a mouse, a keyboard, a touchscreen, a button, etc.) and at least one output device 314 (e.g., a display such as the display 102, speakers, etc.) coupled to the interface 310.

The present disclosure has been described in terms of one or more exemplary embodiments, and it should be appreciated that many equivalents, alternatives, variations, and modifications, aside from those expressly stated, are possible and within the scope of the disclosure.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Reference throughout this specification to “one embodiment” or “an embodiment” may mean that a particular feature, structure, or characteristic described in connection with a particular embodiment may be included in at least one embodiment of claimed subject matter. Thus, appearances of the phrase “in one embodiment” or “an embodiment” in various places throughout this specification is not necessarily intended to refer to the same embodiment or to any one particular embodiment described. Furthermore, it is to be understood that particular features, structures, or characteristics described may be combined in various ways in one or more embodiments. In general, of course, these and other issues may vary with the particular context of usage. Therefore, the particular context of the description or the usage of these terms may provide helpful guidance regarding inferences to be drawn for that context.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent devices by those of ordinary skill in the art. Rather, it is hereby intended the scope be defined by the claims appended hereto. Additionally, the features of various implementing embodiments may be combined to form further embodiments. As used herein, the word “exemplary” means serving as an example, instance, or illustration. Any aspect or embodiment described herein as “exemplary” is not necessarily to be construed as preferred or advantageous over other aspects or embodiments.

1. A fluid measurement device comprising:
(a) a container configured to contain a volume of fluid, the container defining an inlet and an outlet;
(b) a plurality of sensors operatively coupled to the container, the plurality of sensors configured to detect a fluid level within the container; and
(c) a processing device operatively coupled to the plurality of sensors, the processing device configured to process data transmitted by the plurality of sensors to determine at least one rate-based property relating to the fluid.

2. The fluid measurement device of claim 1 wherein at least one sensor of the plurality of sensors is configured to detect at least one property of the fluid within the container.

3. The fluid measurement device of claim 1 wherein the plurality of sensors are coupled to an inner surface of the container.

4. The fluid measurement device of claim 3 wherein the plurality of sensors comprises:
(i) a first array of sensors positioned at a first location of the inner surface of the container, and
(ii) a second array of sensors positioned at a second location on the inner surface of the container different from the first location.

5. The fluid measurement device of claim 4 wherein a first sensor in the first array of sensors and a corresponding first sensor in the second array of sensors are aligned at an equal distance from a first edge of the container.

6. The fluid measurement device of claim 1 wherein the processing device is further configured to periodically release the fluid from the container.

7. The fluid measurement device of claim 1 wherein the processing device is further configured to dynamically adjust an operation of the fluid measurement device based at least in part on a past determination of the at least one rate-based property.

8. The fluid measurement device of claim 1 further comprising a biosensor operatively coupled to the container, the biosensor configured to detect a biomarker in the fluid.

9. A method comprising:
(a) collecting a volume of fluid in a container;
(b) detecting by one or more sensors the volume of fluid in the container; and
(c) determining by a processing device coupled to the one or more sensors at least one rate-based property relating to the volume of fluid using sensor data transmitted from the one or more sensors.

10. The method of claim 9 further comprising:
(a) dynamically adjusting a collected volume of fluid to enable measurements of both low and high fluid flow rates; and
(b) analyzing a time series of rate-based property measurements to determine an abnormal rate value by a heuristics-based algorithm based on a past history of rate values.

11. The fluid measurement device of claim 1 further comprising a distal valve positioned at the outlet at a first end portion of the container, the distal valve movable between a closed position to prevent the fluid from exiting the container and an open position to allow the fluid to be released from within the container.

12. The fluid measurement device of claim 11 further comprising a proximal valve positioned at a second end portion of the container opposite the first end portion, the proximal valve movable between a closed position to prevent the fluid from entering the container and an open position to allow the fluid to enter the container.

13. The fluid measurement device of claim 12 wherein the processing device is further configured to control each of the distal valve and the proximal valve to facilitate releasing the fluid from within the container.

14. A fluid measurement device comprising:
(a) a container configured to contain a volume of fluid, the container defining an inlet and an outlet;
(b) a proximal valve positioned at the inlet of the container, the proximal valve movable between an open position providing fluid communication between a device input tubing and the container and a closed position to prevent fluid from flowing into the container;
(c) a distal valve positioned at the outlet of the container, the distal valve movable between a closed position to retain the fluid within the container and an open position to allow the fluid to exit the container; and
(d) a processing device operatively coupled to the proximal valve and the distal valve to control dispensing of the fluid from within the container.

15. The fluid measurement device of claim 14 wherein the processing device is configured to coordinate opening and closing the distal valve and the proximal valve to facilitate the fluid exiting the container.
